(19) United States
(12) Patent Application Publication
(10) Pub. No.: US 2020/0381131 A1
(43) Pub. Date: Dec. 3, 2020

(54) SYSTEM AND METHOD FOR HEALTHCARE COMPLIANCE

(71) Applicants: Chakravarthy Toleti, Windermere, FL (US); Nageshwara Rao Vempaty, Saratoga, CA (US)

(72) Inventors: Chakravarthy Toleti, Windermere, FL (US); Nageshwara Rao Vempaty, Saratoga, CA (US)

(21) Appl. No.: 16/175,183

(22) Filed: Oct. 30, 2018

(51) Int. Cl. G16H 70/20; G06N 20/00

(52) U.S. Cl. CPC G16H 70/20; G06N 20/00

(57) ABSTRACT

A system and method for managing compliance in healthcare protocols is provided. A plurality of sensors and microphones monitor an environment and generate a plurality of output signals. An analysis subsystem receives the plurality of output signals from the plurality of sensors and microphones. An AI and machine learning subsystem compare the plurality of output signals with a dynamic database of healthcare protocols while a rating system determines a rating corresponding to a level of adherence to the dynamic database of healthcare protocols. An alert system generates an alert corresponding to the level of adherence.

### TECHNICAL FIELD

The embodiments presented relate to a system for three-dimensional interaction tracking of a patient to ensure compliance in a healthcare setting.

### BACKGROUND

The healthcare field relies on the adherence to a variety of protocols outlining standards of care and best practices. Hospitals, nursing homes, private homes, and patient centered homes each provide environments wherein a concert of activity between the patients and care providers must be carefully planned and executed to ensure proper care is provided. These protocols are intended to reduce the likelihood of adverse events.

Many procedures benefit from adherence to a protocol detailing the standard of care and best practices. In one example, modern medicine has benefitted greatly from the discovery of bacteria and the antiseptic procedures used to limit the probability of a potentially life-threatening infection. Despite this, visitors of a patient continue to forget or disregard handwashing protocols when interacting with a patient.

Similarly, more complex care plans require more in-depth monitoring to ensure best practices are implemented. In another example, a patient who has undergone a hip surgery is at risk of pressure ulcers if they remain sedentary for an extended period of time. In current healthcare practices, a medical professional must ensure the patient is turning their body or otherwise moving to prevent a pressure ulcer from forming. Further, this patient may be administered various medications which cause constipation or lack of appetite. Caregivers must ensure the patient is eating at a pre-determined schedule and emptying their bowels regularly, requiring additional hands-on interactions and record keeping by the caregiver. These are often codified as best practices. It is known in the art that such best practices improve quality and consistency of health care provided across the spectrum of care.

In current medical practices, some advanced techniques have been developed to ensure compliance with various protocols. These systems focus on single variable sensing to confirm that a caregiver has visited the patient, such the use of a radio-frequency identification (RFID) tag. These systems do not aggregate data input from a variety of sensors, let alone those which are in direct communication with the patient (e.g., specialized hospital beds and door alarms).

### SUMMARY OF THE INVENTION

This summary is provided to introduce a variety of concepts in a simplified form that is further disclosed in the detailed description of the invention. This summary is not intended to identify key or essential inventive concepts of the claimed subject matter, nor is it intended for determining the scope of the claimed subject matter.

The present embodiments disclose a system and method for managing compliance in healthcare protocols. A plurality of sensors and microphones monitor an environment and generate a plurality of output signals. An analysis subsystem receives the plurality of output signals from the plurality of sensors and microphones. An Artificial Intelligence (AI) and machine learning subsystem compare the plurality of output signals with a dynamic database of healthcare protocols while a rating system determines a rating corresponding to a level of adherence to the dynamic database of healthcare protocols. An alert system generates an alert corresponding to the level of adherence.

In one aspect, the alert includes an audio alert transmitted to a plurality of speakers in the environment.

In one aspect, the analysis subsystem is configured to identify one or more objects and one or more agents in the environment using an identifier disposed on each of the one or more objects and each of the one or more agents. The identifier utilizes at least one of the following means: a thermal tag, an Infrared (IR) tag, identity badge, shape recognition, or an RFID.

In one aspect, the plurality of sensors comprises one or more thermal imaging cameras.

In one aspect, the AI and machine learning subsystem includes a dynamic configuration module configured to provide updated data to the dynamic database of healthcare protocols. The rating system compares an outcome of an event or a behavior to an expected outcome of the dynamic database of healthcare protocols, to detect if the outcome may be considered an anomaly in an event or a behavior.

In one aspect, a method for determining compliance in healthcare protocols comprises the steps of receiving, via an AI and machine learning subsystem, a plurality of output signals each corresponding to an event. The output signals are generated by a plurality of sensors and a plurality of microphones, each of the plurality of output signals corresponding to one or more agents in an environment. Next a rating is determined via a rating system in operable communication with the AI and machine learning subsystem. The rating is compared, via the AI and machine learning subsystem, to a plurality of defined ratings for a reference pattern stored in a dynamic database of healthcare protocols. An alert is generated if an anomaly is found.

### BRIEF DESCRIPTION OF THE DRAWINGS

A complete understanding of the present invention and the advantages and features thereof will be more readily understood by reference to the following detailed description when considered in conjunction with the accompanying drawings wherein:

FIG. 1 illustrates a schematic of a healthcare environment, according to some embodiments;

FIG. 2 illustrates a block diagram of the healthcare protocol compliance monitoring system, according to some embodiments;

FIG. 3 illustrates a flowchart of the healthcare compliance monitoring system and method for dynamically updating protocols, according to some embodiments;

FIG. 4 illustrates a flowchart for preprocessing for protocol rating and behavioral analysis systems, according to some embodiments;

FIG. 5A illustrates a flowchart of the activity detection system, according to some embodiments;

FIG. 5B illustrates a flowchart of the voice command system, according to some embodiments; and

FIG. 6 illustrates a block diagram of the analysis subsystem, according to some embodiments.

### DETAILED DESCRIPTION

The specific details of the single embodiment or variety of embodiments described herein are to the described system and methods of use. Any specific details of the embodiments are used for demonstration purposes only and not unnecessary limitations or inferences are to be understood therefrom.

Before describing in detail exemplary embodiments, it is noted that the embodiments reside primarily in combinations of components related to the system and method. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like, may be used solely to distinguish one entity or element from another entity or element without necessarily requiring or implying any physical or logical relationship or order between such entities or elements.

The system and method disclosed herein provide for an automated sensor and reasoning system which monitors protocol adherence and detects the broach of the protocol to alert a caregiver of the noncompliant breach appropriately. The system and method further store data related to both the compliance or any breach of a variety of protocols to aggregate data which can be used to modulate the protocols for future improvements automatically. As a result the system and method act to reduce adverse patient events by improving caregiver and patient adherence to the best practices and standard of care defined by the protocol.

While the detailed description focuses on “caregivers,” a skilled artisan will recognize that the system and method may be applied to any person interacting with the patient, including visitors for example. Further, while the system and method set forth herein are described with respect to a healthcare clinical process, it can be readily appreciated that the system and method are equally applicable to a variety of human activities that involve human interaction with a predefined protocol to achieve an objective, such as, for example, manufacturing, food preparation, apparatus service, training, customer service, security, etc. The term “agent” refers to any person which the system is sensing and monitoring. An agent can include caregivers such as doctors, lab technicians, nurses, and transportation staff as well as non-caregivers such as the patient or visitors.

A sensor system is positioned to monitor the behavior of persons in an environment. Each sensor gathers data and transmits this data to a computer vision subsystem. The computer vision subsystem executes various algorithms to analyze the behavior and interactions between persons and objects in the environment. The analysis of behavior is utilized to determine adherence to a pre-defined protocol. Detection of objects, agents, and the interactions thereof is permitted via the input from multiple sensors, the number of which is configurable and not necessarily fixed. Embodiments of the invention use a multi-camera, multi-object, and multi-agent tracking system that includes a number of software subsystems which are used to characterize events, activities, and behaviors, as well as determine the identification of objects and agents in an environment.

In some embodiments, a goal of the system and method is to ensure adequate patient care is provided in view of the predefined protocols stored in the system. If protocols are not adhered to, a caregiver is notified by an alert system to modify the behavior of the caregiver in future interactions. The behavior of the caregivers, visitors, and patients alike are monitored and notifications and alerts to modify behavior are provided to ensure the standard of care is realized.

In some embodiments, a goal of the system and method is to provide continuous monitoring throughout the process of care to a patient. The intent of the system and method is to analyze the behavior of the caregiver and patient alike while predicting breaches in pre-determined protocols. Data is aggregated and compared with the outcome of the care which has been provided. This data can be used to modify pre-existing protocols in real-time and to ensure optimal care is provided to each specific patient.

As used herein, the term “protocol” refers to an ordered sequence of events and tasks performed by one or more persons. Using healthcare as an example, protocols can include tasks, clinical techniques and events, policies, regulatory events, administrative events, and likewise tasks which are specific to each patient. In one example, a protocol can be exemplified in the behavior of a caregiver washing his or her hands before interacting with a patient.

Sensors can include optical sensors such as video cameras, infrared cameras, thermal imaging cameras, motion sensors, audio sensors (microphones), pressure sensors and pressure sensors disposed throughout the environment to analyze a viewing field. Further, medical sensing devices commonly found in hospitals, skilled nursing facilities, hospice facilities, and other patient care environments can be in communication with the system.

The computer vision subsystem can include an analysis module for anomaly detection with respect to the pre-defined protocol. This analysis module can utilize a comparator to compare data received from the sensing elements. The computer vision subsystem may define an acceptable range of data values from the pre-defined protocol while the comparator compares the received values to the predefined acceptable range. A data value outside of this range (i.e., an anomaly) may trigger a message, alert, or likewise notification from the alert subsystem.

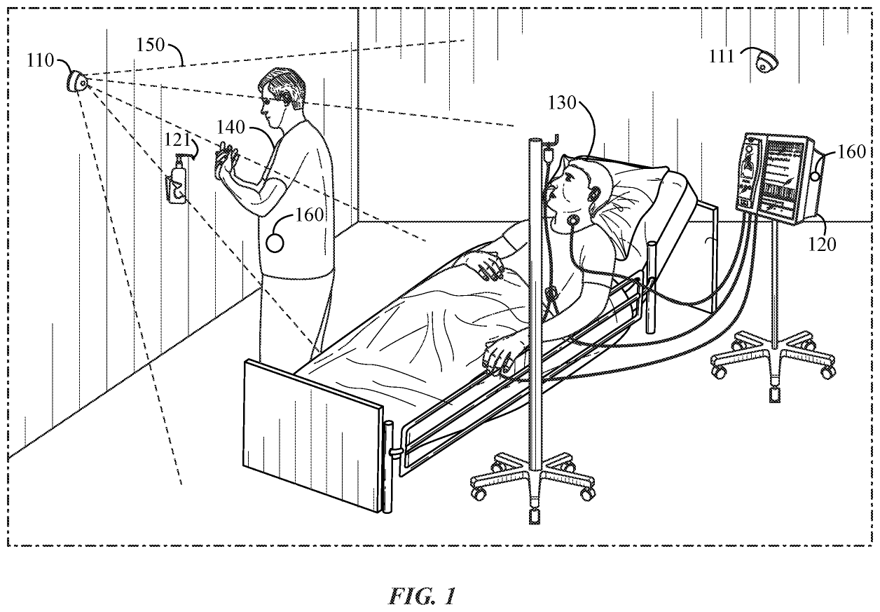

FIG. 1 illustrates an exemplary schematic of a system for monitoring protocol compliance. The variety of people and objects presented are relative to the task of the protocol, allowing one skilled in the arts to understand the variety of persons and objects to be utilized in each situation and environment. As illustrated, the environment 100 includes a plurality of sensors 110, 111 positioned to monitor behaviors of objects 120, patients 130, and caregivers 140.

In some embodiments, the sensor 110 is an optical sensor having a field-of-view 150 which includes the various objects 120, patients 130, and caregivers 140 within the environment 100 such that the behaviors and interactions of each can be sensed. The sensor 110 can be used to identify objects 120, devices 121, patients 130, and caregivers 140 within the environment 100 by sensing an identifier 160. In one aspect, a unique identifier 160 is positioned on one or more objects 120, patients 130, or caregiver 140 such that a determination can be made toward the identity and the relevance to the pre-defined protocol.

In some embodiments, facial recognition or shape analysis and recognition is used to identify the objects 120, devices 121, patients 130, and caregivers 140 in the environment.

The device 121 is illustrated as a hand sanitizer. In some embodiments, the device 121 can include any device utilized in the environment, including a device 121 used during a healthcare protocol. The device can include a sink, soap, gloves, or other medical devices commonly used during a healthcare protocol.

In some embodiments, the identifier 160 can include a thermal tag which may be unique to each object 120, patient 130, and caregiver 140.

Sensor 110 can be positioned to monitor dynamic signals read from the displays of objects 120 such as, for example, an EKG, a medication, a food, or similar clinical devices in the environment 100. In another example, the operation status of objects 120 such as the sink and knobs thereof, the soap, or battery level indicators can be monitored by the sensor 110. Sensors 110 can be configured to monitor non-medical devices having pre-existing sensors, including bed position, bedding components, doors, windows, lights, or similar objects found in the environment 100. Each output produced by the signal is transmitted to a computer vision subsystem and components therein.

In some embodiments, the sensor 110 includes a HIPAA compliant camera (whether optical, infrared, thermal, or likewise) such that data output from the sensor 110 is of an event rather than raw image data. The data transmission can include each object 120, patient 130, and caregiver 140 within the environment 100 by identifying each using each of their respective unique identifiers 160 (whether a thermal tag, infrared tag, RFID, identity badge, or shape recognition). The activity performed is inferred by the computer vision subsystem. If no determination or identifications can be made, image data can be transmitted to a remote monitoring system.

In some embodiments, the environment can include a plurality of rooms such as for example, a bedroom, bathroom, entry/exit way, and hallway. Sensors 110 can be positioned in each room and each multiple view areas of each room. The agent's movement can be monitored between each room in the environment 100 such that the system can create a cohesive event stream across the variety of rooms.

In an alternate embodiment, a mobile sensor 110 may enter or exit the room on a pre-determined schedule to monitor behaviors and activities.

In some embodiments, auxiliary sensor 111 can include various sensors known in the arts of healthcare monitoring. Auxiliary sensor 111 can be used in tandem with sensor 110.

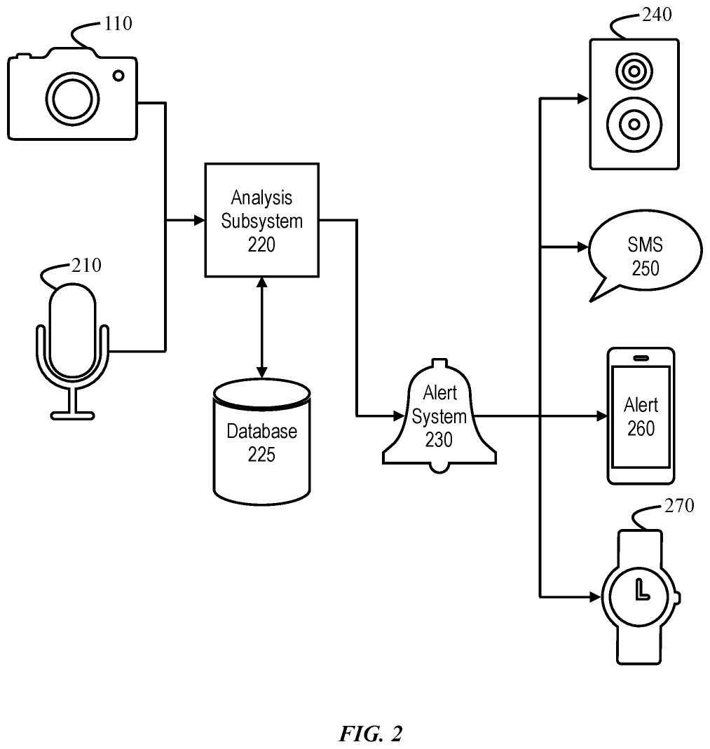

FIG. 2 illustrates an exemplary flowchart showing information and data flow through the system for healthcare compliance. One or more sensors 110 which can include at least one microphone 210 to monitor audio commands given by any agent such as the patient 130, or caregiver 140. The microphone 210 can send an output signal to the analysis subsystem 220 to determine if an action such as an alert is optioned by the pre-defined protocol.

An analysis subsystem 220 receives sensor 110 and microphone 210 output signals. The analysis subsystem 220 collects, aggregates, and analyzes the outputs signals to determine if appropriate action is needed for each event within the environment 100. The analysis subsystem can include a comparator to compare output signal data with historical data related to each pre-defined protocol.

Upon the detection of an anomaly by the analysis subsystem 220, a response signal can be sent to an alert system 230. The alert system 230 can include a speaker 240 in an audible range of at least one of the agents within the environment 100 or at a remote location. In some embodiments, the alert system 230 can generate various types of alerts including short messaging system (SMS) alerts 250 to a plurality of agents, alerts to pre-existing notifications systems 260 in the environment 100, or likewise means for receiving alerts by caregivers and the hospital. Further, a smartwatch 270 and similar smart devices can receive audio or visual alerts as known in the arts.

In some embodiments, alerts generated by the alert system 230 may be assigned a priority level which determines an escalation pattern for each alert. In one example, a high-priority alert can result in an SMS alert 250 to a caregiver 140 near the patient 130.

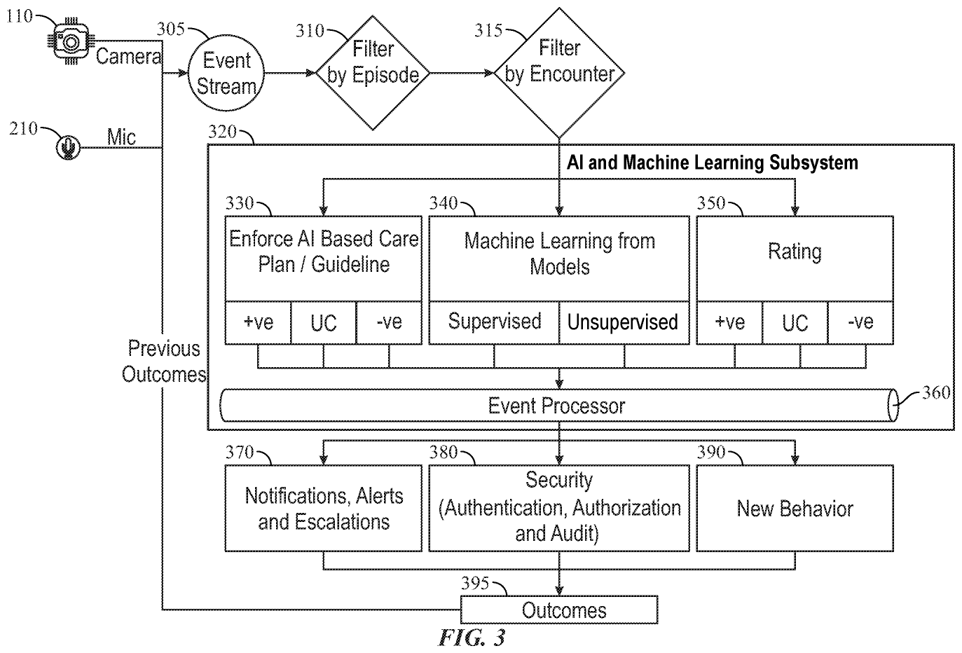

FIG. 3 illustrates a flowchart of a system and method for classifying behaviors and interactions between various agents in an environment 100. As described herein, sensors 110 and microphones 210 continuously monitor the environment 100, in combination with prior outcomes stored in the database 225, to collect a plurality of event streams as shown in block 305. Each event stream 305 is filtered by one or more parameters such as for example the type of interaction, agents involved, and time period of the interaction as shown in blocks 310 and 315. Filtered event streams are transmitted to the analysis subsystem 220 which can include an artificial intelligence (AI) and machine learning subsystem 320. In block 330, the analysis subsystem consults an AI-based protocol and compares the signal outputs from the sensors 110 to the AI-based protocol values. The analysis subsystem 220 performs machine learning from models stored in the database 225 in block 340 and provides a rating in block 350.

In some embodiments, the AI and machine learning subsystem 320 utilize deep learning and machine learning concepts such as deep neural networks (DNN) and recurrent neural networks (RNN) to perform various analyses of the environment 100 including the objects 120, patients 130, and caregivers 140 (collectively referred to as agents) therein. Event recognition can be supervised or unsupervised. Once a determination of the event/activity has been made, the event/activity can be categorized as safe or unsafe depending on the presence or absence of an anomaly. The category (safe or unsafe) determination is utilized to rank and rate behaviors and interactions to provide feedback to the agents of the environment 100.

In some embodiments, an event processor 360 receives an event signal from the AI and machine learning subsystem 320 and sends an output signal to the alert system 230. The alert system can provide notifications, alerts, and escalations as shown in block 370, a security alert as shown in block 380, and a new behavior notification as shown in block 390. Each output from the system 370, 380, 390 is stored as an outcome 395 and stored in the database 225 to be consulted in future event streams.

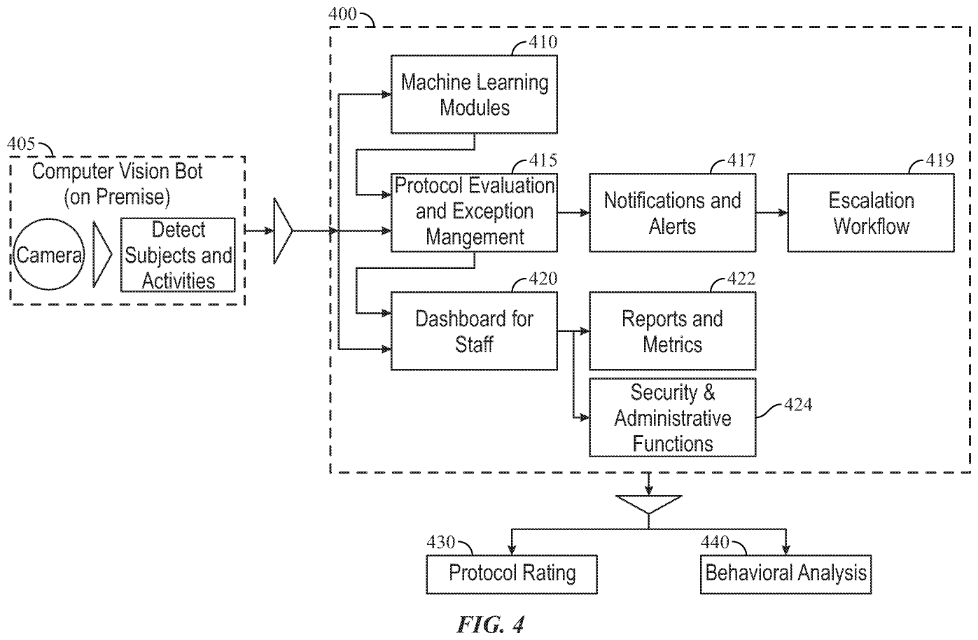

FIG. 4 shows an exemplary workflow of the system for monitoring healthcare compliance. Sensors 110, such as a camera detects activities, behaviors, and agents within an environment 100 to generate an output signal having the classification of the behavior and event data as shown in block 405. In some embodiments, the output signal does not contain images, video, or audio to preserve privacy. The output signal is provided to the server 400 which includes AI and machine learning modules (block 410), protocol evaluation modules (block 415), and a user dashboard (block 420).

The protocol evaluation modules (block 415) manage the output of various notifications and alert (block 417) in addition to the escalation of the alerts and notifications (block 419). The dashboard (block 420) provides a user interface to generate, view, or analyze reports, metrics, and perform security or administrative functions as shown in blocks 422 and 424. A protocol rating 430 and behavior analysis 440 is generated by the analysis subsystem 220 to inform future behaviors and interactions within the environment 100.

In some embodiments, the AI machine learning modules 410 utilize deep neural networks, which can include convolutional neural networks and recurrent neural networks to learn and infer events and behaviors from the incoming event stream 305. The protocol evaluation modules 415 process the events and behaviors received from the AI and machine learning modules 410 to compare the behavioral and activity data from the output signal, to the stored pre-defined protocols. The commonalties and differences are compared and scored by a comparator to determine if a safety concern is detected. The detection of a safety concern can be transmitted to as an alert signal to an escalation management module in communication with the alert system 230 for further processing. Further processing can include the generation of an alert or an escalation.

In some embodiments, a user dashboard module displays the events on a user interface enabling agents, such as a caregiver, to view real-time, near real-time, or previous views of the event stream 305.

In some embodiments, sensors 110 are triggered by a motion event within the environment 100. This can include object 120, patient 130, or caregiver 140 movement. In one example, in the event that a patient 130 falls out of bed, the sensor 110 recognized the motion and begins procedures as described hereinabove. This can include analyzing the fall distance of the patient 130 and movement, or lack thereof, following the fall event. If the patient 130 is non-responsive, the alert system 230 can generate an alert signal including an audio alert utilizing the speaker 240, for example, to ask if the patient is okay. If no response is detected via the sensor 110 or microphone 210, the alert is escalated by sending an SMS alert 250 to a nearby caregiver 140. The SMS alert 250 determines a recipient (the caregiver 140) and associates an identifier 160 with the recipient. The sensor 110 can detect the entrance of the caregiver 140 and confirm the identifier 160. The interaction between the caregiver 140 and the patient 130 is continuously monitored until an outcome 395 is reached. In the particular example, an outcome 395 can include the patient 130 returning to his or her bed.

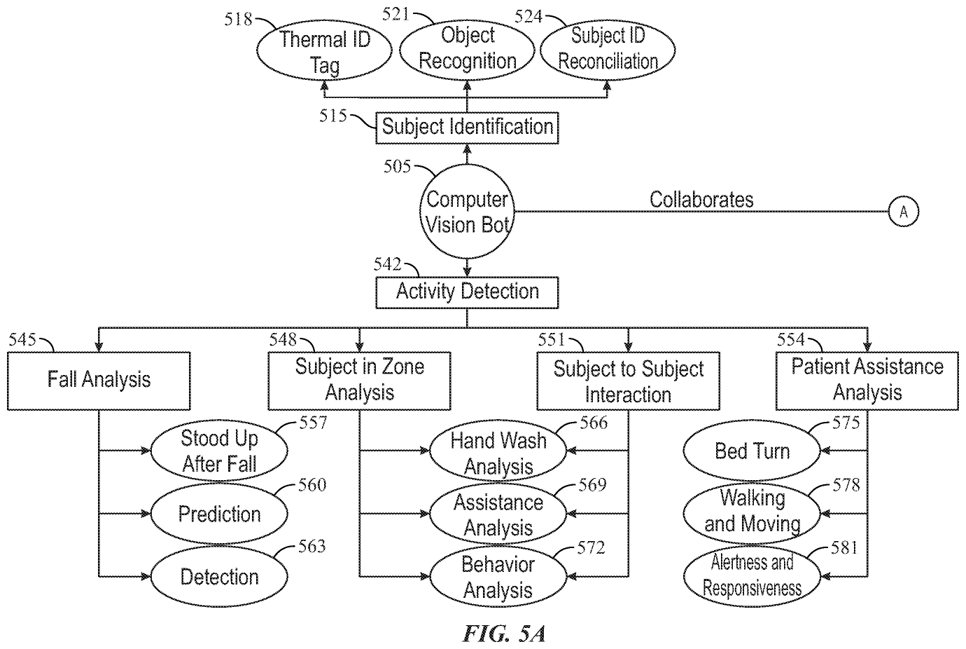

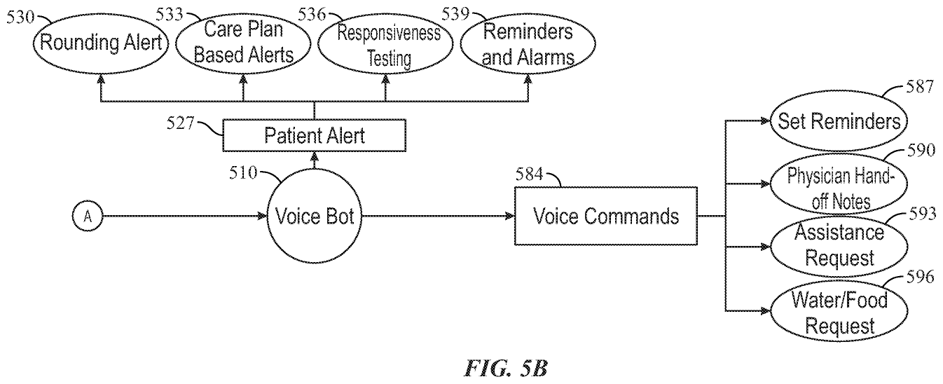

FIG. 5 illustrates an exemplary flowchart of the event and behavior classification system. In blocks 505 and 510, the computer vision subsystem which includes the analysis subsystem 220 collaborates with the microphones 210, speakers 240, and alert system 230. The analysis subsystem 220 performs subject identification in block 515 which can include identifier 160 recognition which may be placed on an agent or object 120 or similar identification procedures (blocks 518, 521, 524). The alert system 220, in communication with the speaker 240 communicates a patient alert in block 527. Blocks 530, 533, 536, and 539 include alert types including rounding alerts, care plan-based alerts, responsiveness testing, and reminders and alarms. Further, the alert system 220 and speaker 240 generate, send, and receive voice commands in block 584. Voice commands can include a recording of reminders in block 587, caregiver notes in block 590, and various requests by one or more agents in block 593 and 596.

In some embodiments, each alert is a dynamic response to an output signal and audio signal in the environment. In such, if an alert is generated by the patient falling out of bed, a dynamic response can ask if the patient injured. Depending on the patient's response of “yes” or “no”, various alert cascades can be dynamically generated.

Block 542 includes activity detection performed by the analysis subsystem 220. Activity detection can include, by way of example fall detection (block 545), subject location analysis (block 548), interaction analysis (block 551), and patient assistance analysis (block 554).

In some embodiments, fall analysis includes fall prediction (block 560), fall detection (block 563), and events following the fall, such as whether or not the patient stood up (block 557). Subject location analysis and interaction analysis can include hand washing analysis (block 566), assistance analysis (block 569), and behavior analysis (block 572).

In some embodiments, patient assistance analysis can include bed turn recognition (block 575), movement recognition (block 578), and alertness and responsiveness recognition (581).

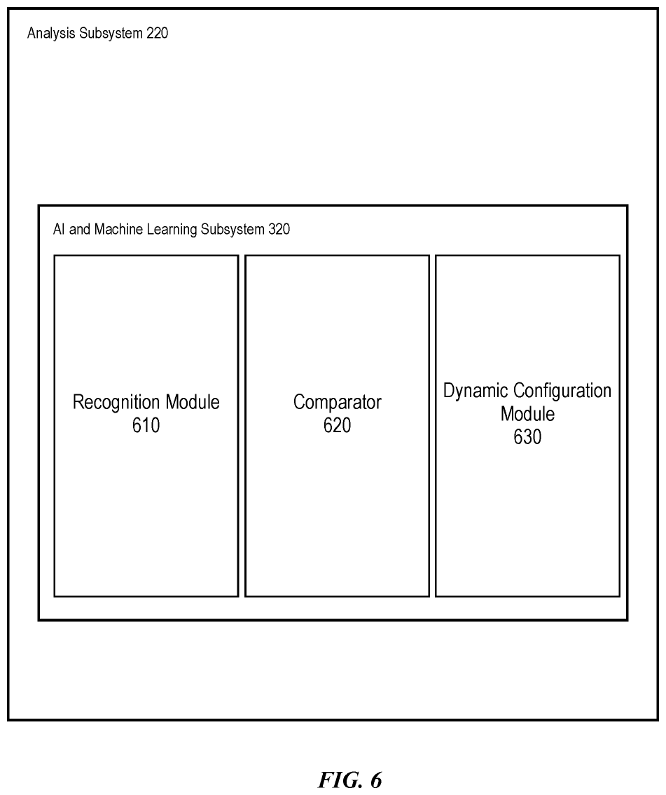

In reference to FIG. 6, identifier 160 recognition is performed by a recognition module 610 which can be provided as a component of the analysis subsystem 220. The recognition module 610 utilizes identifiers 160 (such as thermal tags) provided on the objects 120 or the agents. Comparator 620 can be utilized to compare images of objects 120 to objects 120 present in the field-of-view 150. In alternate embodiments, identifiers 160 can be positioned on the objects 120.

In some embodiments, gait analysis is performed using pre-existing gait analysis data to determine patient behavior. Gait analysis can be used to predict, detect and analyze a fall.

Subject location analysis compares activity and behavior of the subject (which can include an agent) against pre-defined activities and behaviors relevant to a specific location. For example, an agent located near a hand washing station may be expected to wash his or her hands. The activity and behavior of the subject are then analyzed by the analysis subsystem 220 to determine if the agent washed their hand or not. Interactions between subjects can include interactions between an object and an agent. Identifiers 160 are used to determine an identity of objects 120, patients 130, and caregivers 140.

In some embodiments, a dynamic configuration module 630 allows for the AI and machine learning subsystem 320 to dynamically update information in the database 225. The updated information can relate to protocols, standards of care, best practices, acceptable rating thresholds, or other information related to events, behaviors, and activities occurring in the environment.

In some embodiments, patient assistance utilized the microphones 210 and alert subsystem 230 to analyze patient voice commands and alert agents to respond to the patient request.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or suhcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for managing compliance to healthcare protocols, the system comprising:
a plurality of sensors configured to monitor an environment and generate a plurality of output signals;
a plurality of microphones configured to monitor the environment and generate respective audio data;
an analysis subsystem to receive the plurality of output signals from the plurality of sensors and the audio data from the plurality of microphones;
an AI and machine learning subsystem to compare the plurality of output signals and the plurality of audio data with a dynamic database of healthcare protocols, the AI and machine learning subsystem are configured to dynamically respond to the plurality of output signals;
a rating system to determine a rating corresponding to a level of adherence to the dynamic database of healthcare protocols; and
an alert system to generate an alert corresponding to the level of adherence to the healthcare protocol.

2. The system of claim 1, wherein the alert includes an audio alert transmitted to a plurality of speakers in the environment, wherein the audio alert dynamically responds to the plurality of output signals.

3. The system of claim 1, wherein the analysis subsystem is configured to identify one or more objects and one or more agents in the environment using an identifier disposed on each of the one or more objects, and each of the one or more agents.

4. The system of claim 3, wherein the identifier utilizes at least one of the following means: a thermal tag, an IR tag, facial recognition, identity badge, shape recognition, or an RFID.

5. The system of claim 1, wherein the plurality of sensors comprises one or more thermal imaging cameras.

6. The system of claim 1, wherein the AI and machine learning subsystem includes a dynamic configuration and learning module to provide updated data to the dynamic database of healthcare protocols.

7. The system of claim 6, wherein the rating system compares an outcome of an event or behavior to an expected outcome of the dynamic database of healthcare protocols, wherein the outcome corresponds to an anomaly in an event or behavior.

8. A system for determining compliance in healthcare protocols, the system comprising:
a plurality of sensors configured to monitor an environment having one or more agents and generate a plurality of output signals;
a plurality of microphones configured to monitor the environment and receive audible commands from one or more agents;
an analysis subsystem to receive the plurality of secure output signals from the plurality of sensors and the audio data from the plurality of microphones;
an AI and machine learning subsystem configured to receive and compare the plurality of output signals and the plurality of audio data with a dynamic database of healthcare protocols, the AI and machine learning subsystem configured to enforce the dynamic database of healthcare protocols via a rating system configured to generate a rating corresponding to a level of adherence to the dynamic database of healthcare protocols; and
an alert system to generate an alert corresponding to the level of adherence to the dynamic database of healthcare protocols, the alert system in operable communication with a plurality of speakers, and a plurality of devices in dynamic audible or visual communication with at least one of the plurality of agents in the environment.

9. The system of claim 8, wherein the plurality of microphones is in communication with the AI and machine learning subsystem configured to analyze the audio data and generate a response corresponding to the audio data.

10. The system of claim 8, wherein the alert system is in operable communication with one or more pre-existing security systems in the environment.

11. The system of claim 8, wherein the plurality of sensors comprises one or more thermal imaging cameras.

12. The system of claim 11, wherein the thermal imaging cameras transmit an event stream comprising secure thermal imaging data, wherein the secure thermal imaging data is filtered by at least one parameter.

13. The system of claim 8, wherein the AI and machine learning subsystem includes a dynamic configuration module configured to provide updated data to the dynamic database of healthcare protocols.

14. The system of claim 13, wherein the rating system compares an outcome of an event or behavior to an expected outcome of the dynamic database of healthcare protocols, wherein the outcome corresponds to an anomaly in an event or behavior, and wherein the anomaly results in the generation of the alert via the alert subsystem.

15. The system of claim 8, wherein the analysis subsystem is configured to identify one or more objects and one or more agents in the environment using an identifier disposed on each of the one or more objects, and each of the one or more agents.

16. A method for determining compliance in healthcare protocols, the method comprising:
receiving, via an AI and machine learning subsystem, a plurality of output signals each corresponding to an event, the output signals generated by a plurality of sensors and a plurality of microphones, the plurality of output signals corresponding to one or more agents in an environment;
determining, via a rating system in operable communication with the AI and machine learning subsystem, a rating for each of the plurality of output signals;
comparing, via the AI and machine learning subsystem, the rating to a plurality of defined ratings for a reference event stored in a dynamic database of healthcare protocols;
generating a dynamic alert if the rating is an anomaly compared to the reference event.

17. The method of claim 15, wherein the dynamic alert system is in operable communication with one or more pre-existing security systems in the environment.

18. The method of claim 15, wherein the AI and machine learning subsystem includes a dynamic configuration module configured to learn and provide updated data to the dynamic database of healthcare protocols.

19. The method of claim 15, wherein the plurality of sensors comprises one or more thermal imaging cameras.

20. The method of claim 15, wherein pre-existing sensors in the environment are in communication with AI and machine learning subsystem.
